(19) United States
(12) Patent Application Publication
(10) Pub. No.: US 2007/0260309 A1
(43) Pub. Date: Nov. 8, 2007

(54) Accommodating intraocular lens having a recessed anterior optic

(76) Inventor: Gary A. Richardson, Rochester, NY (US)

Correspondence Address: Bausch & Lomb Incorporated, One Bausch & Lomb Place, Rochester, NY, 14604-2701, US

(21) Appl. No.: 11/429,887

(22) Filed: May 8, 2006

(51) Int. Cl. A61F 2/16

(52) U.S. Cl. 623/6.34; 623/6.37

(57) ABSTRACT

A multi-component accommodating intraocular lens (AIOL), comprising a recessed anterior optical element, a posterior component, and at least two longitudinal haptics each coupled to at least a portion of the anterior optic and at least a portion of the posterior component. Each haptic has an anterior most-portion that is disposed more anteriorly than a corresponding portion of an edge of the anterior optical element.

### FIELD OF INVENTION

The present invention relates to accommodating intraocular lenses (AIOLs), and more particularly to AIOLs having a recessed anterior optic.

### BACKGROUND OF THE INVENTION

FIG. 1 illustrates a cross-sectional view of a human eye 10 having an anterior chamber 12 and a posterior chamber 14 separated by an iris 30. Within the posterior chamber 14 is a capsular bag 16 which holds the eye's natural crystalline lens 17. Light enters the eye by passing through cornea 18. The cornea and crystalline lens act together to direct and focus the light onto retina 20. The retina is connected to optic nerve 22 which transmits images received by the retina to the brain for interpretation. Eye 10 has a visual axis VA.

In response to the sharpness of the image received by the retina, the brain operates to contract or relax ciliary muscle 26. Ciliary muscle 26 is disposed within ciliary body 28, and upon contraction of the ciliary muscle, the ciliary body is caused to move. To achieve near focus accommodation, the ciliary muscle is contracted thereby causing the cilliary body to relax tension on zonules 27 which permits the capsular bag and lens 17 to become more rounded. To achieve far focus (i.e., disaccommodation), the ciliary muscle is relaxed thereby increasing tension on zonules 27 which causes the capsular bag and lens 17 to become flatter.

In an eye where the natural crystalline lens has been damaged (e.g., clouded by cataracts), the natural lens is no longer able to properly focus and/or direct incoming light to the retina. As a result images become blurred. A well known surgical technique to remedy this situation involves removal of a damaged crystalline lens through a hole in the capsular bag known as a capsularhexis (also referred to simply as a rhexis). Subsequently, an artificial lens known as an intraocular lens (IOL) can be placed into the evacuated capsular bag through the rhexis.

Conventional IOLs are typically fixed-focus lenses. Such lenses are usually selected to have a power such that the patient has a fixed focus for distance vision, and the patient requires spectacles or contact lenses to permit near vision. In recent years extensive research has been carried out to develop IOLs having variable focus capability. Such IOLs are known as accommodating IOLs (AIOLS). The term “AIOLs” refers to both single-element and multi-element systems.

AIOLs permit a wearer to have accommodative vision. AIOLs are typically located in the posterior chamber (e.g., in the capsular bag) and provide variable focal power in accordance with tension or a lack of tension exerted on the capsular bag 16 as a result of contraction and relaxation of the ciliary muscle. FIG. 2 shows an exemplary two-element IOL 24 in capsular bag 16. IOL 24 comprises an anterior lens element 42 and a posterior lens element 44 that are connected to one another by haptics 46. The haptics permit lens elements 42 and 44 to translate relative to one another to achieve accommodation and disaccommodation. Further details of IOL 24 are given in U.S. Pat. No. 6,488,708 issued Dec. 3, 2002, to Sarfarazi

Problems with AIOLs that have been implanted to date include that (1) they have made contact with the iris of an eye in which they are implanted, (2) they have provided less than desirable amounts of accommodation, and (3) they have acted unpredictably when implanted in an eye. To date no suitable solution for avoiding contact with the iris has been provided and the reason(s) for the lack of accommodation and unpredictability have not been definitively identified.

### SUMMARY OF THE INVENTION

Aspects of the present invention are directed to a multi-component accommodating intraocular lens comprising an anterior optical element, a posterior component, and at least two longitudinal haptics each coupled to at least a portion of the anterior optic and at least a portion of the posterior component. Each haptic has an anterior-most portion that is disposed more anteriorly than an edge of the anterior optical element. As used herein the term “anterior” means toward the front of an eye. As used herein the term “component” is inclusive of any of an optical element (having optical power or no optical power), a frame having an open aperture (i.e., no optical element) or any other structure suitable for maintaining the shape of a capsular bag.

In some embodiments, the AIOL is sized and shaped to conform to the interior surface of a capsular bag of a patient's eye. In some embodiments, the AIOL has an overall diameter of approximately 9.3 mm. In some embodiments, the anterior most-portion of each haptic is disposed approximately 0.75 mm more anteriorly than the anterior side of the corresponding portion of the edge of the anterior optical element.

Each of the haptics may be integrally formed with the anterior optic. In some embodiments, the posterior component comprises an optical element. In some embodiments, the anterior optical element has a positive optical power and the posterior optical element has a negative optical power. The haptics may cover 30-40 degrees of longitude at the anterior lens.

### BRIEF DESCRIPTION OF THE DRAWINGS

Illustrative, non-limiting embodiments of the present invention will be described by way of example with reference to the accompanying drawings, in which the same reference number is used to designate the same or similar components in different figures, and in which:

FIG. 1 is a schematic illustration of a cross-sectional view of a human eye;

FIG. 2 is a schematic illustration of a cross-sectional view of a human eye with a two-element AIOL implanted in the capsular bag;

FIG. 3 is a perspective view of an open chamber, AIOL having three haptics extending between an anterior lens optic and a posterior lens optic in accordance with one embodiment of the invention;

FIG. 4 is an end view of the open chamber AIOL, as depicted in FIG. 3, viewed along an optical axis;

FIG. 5A is a side view of the AIOL as depicted in FIG. 3;

FIG. 5B is a cross-sectional side view of the AIOL as depicted in FIG. 3; and

FIG. 6 is a partial, projection view of the AIOL as depicted in FIG. 3, the AIOL being disposed within a capsular bag.

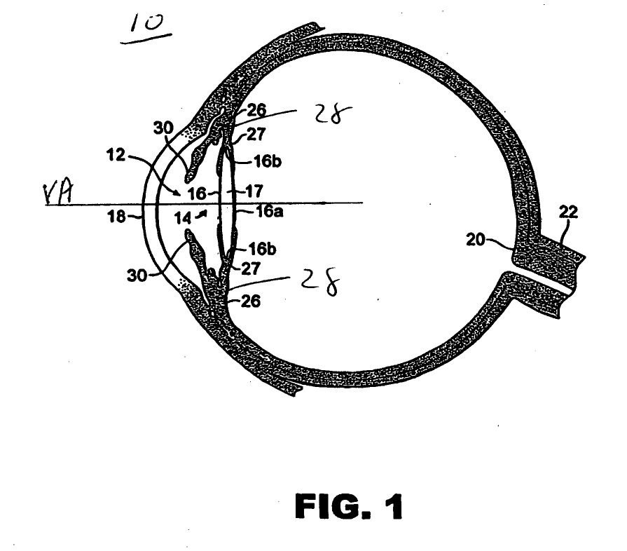

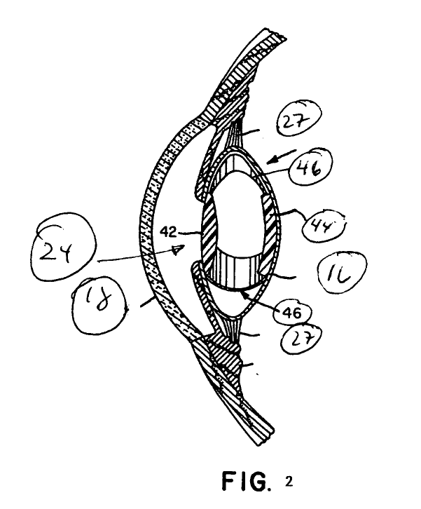

### DETAILED DESCRIPTION

Aspects of the present invention are directed to a multi-component accommodating intraocular lens, comprising: an anterior optical element; a posterior component; and at least two longitudinal haptics each coupled to at least a portion of the anterior optic and at least a portion of the posterior component, each haptic having an anterior-most portion that is disposed more anteriorly than an edge of the anterior optical element. That is to say, the anterior optic is recessed relative to the haptics.

By recessing the anterior optic, the amount of contact between the AIOL and the iris of an eye can be reduced or eliminated. Also, by providing a recessed anterior optic, contact between the capsular bag and the AIOL is made with the anterior-most portion portions of the haptics rather than the haptic and the anterior optic. As a result, the rhexis made in the eye can be larger than with a conventional lens. Experiments using cadaver eyes have indicated that, by providing a larger rhexis, the flexibility of the anterior portion of the capsular bag can be increased, and as a result the amount of forward translation of the anterior optic during accommodation can be increased, thereby increasing the amount of accommodation provided by the AIOL.

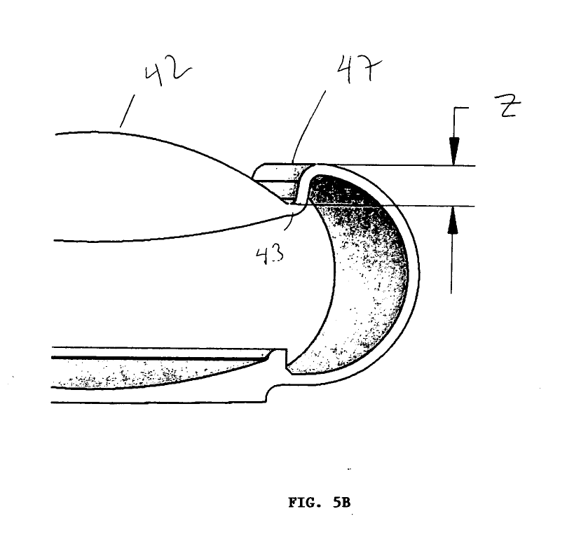

Further, because the capsular bag does not make contact with the anterior surface of the lens, the impact of the size, shape and centricity of the rhexis is reduced. Additionally, the longitudinal arrangement of the haptics that provide for the recessed anterior optic according to aspects of the invention, also provide accommodative movement that occurs along the optical axis of the lenses (without a predisposition to move in other directions) thereby reducing the tendency of the lens to tilt or twist during the accommodative movement. Furthermore, because the lens is recessed, the anterior-most portion of the haptics can be compressed radially inward (i.e., toward the optical axis of the lens, as indicated by R in FIG. 5A) to facilitate placement of AIOLs in capsular bags of different sizes. The compression can occur without a predisposition to tilting or twisting.

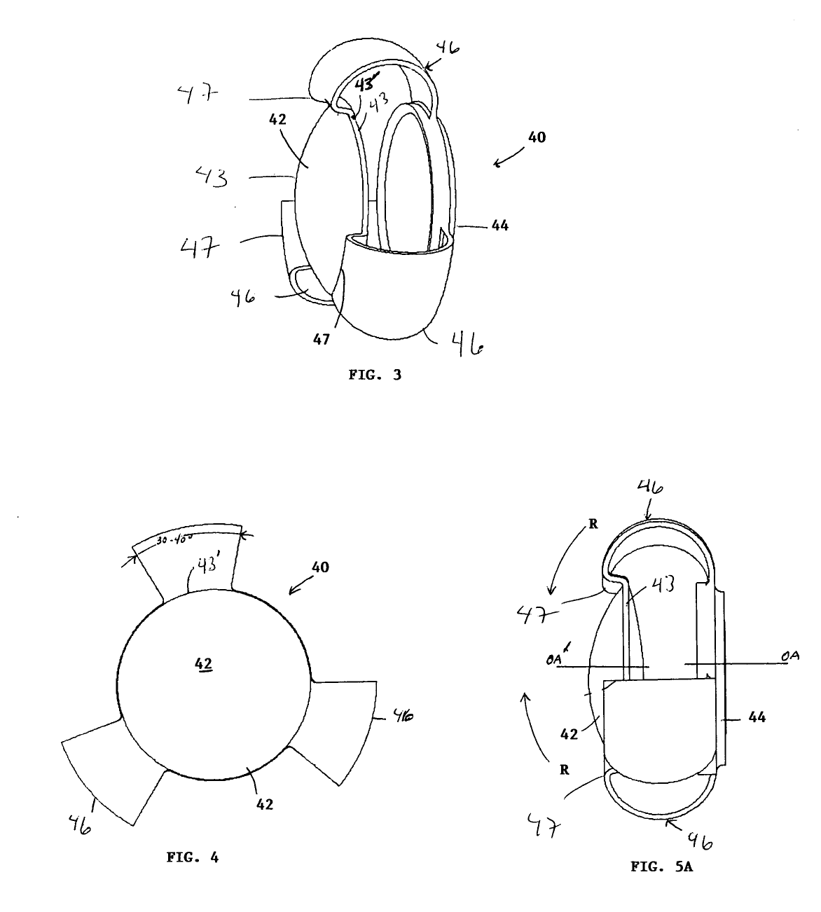

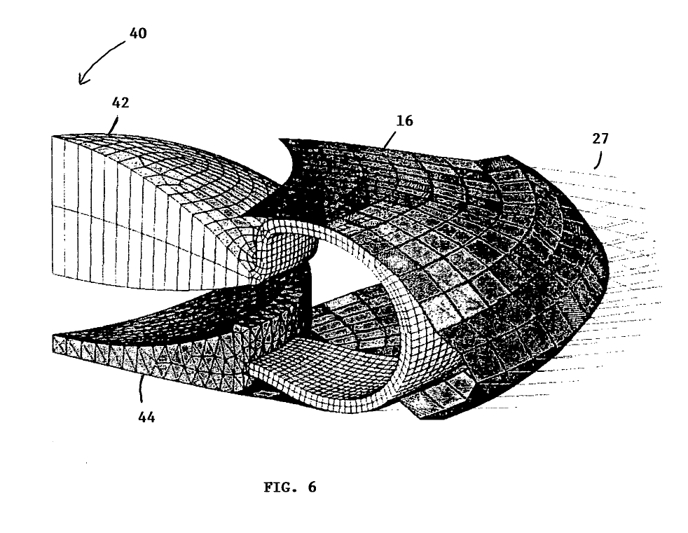

FIG. 3 is a perspective view of a multi-element AIOL 40 according to aspects of the present invention. AIOL 40 comprises an anterior optical element 42; a posterior component 44; and three longitudinal haptics 46 each coupled to at least a portion of the anterior optic and at least a portion of the posterior component. AIOL 24 substantially conforms to the interior surface of the capsular bag 34, other than at the anterior optic. As is apparent in FIG. 6, the rhexis may be made such that the haptics maintain the capsular bag in a position suspended above the anterior optic.

Turning to FIGS. 4 and 5A, there is shown a front view of AIOL 40, and a side view of AIOL 40, respectively. As is apparent in FIG. 5A, each haptic 46 has an anterior-most portion 47 that is disposed more anteriorly than an anterior side of edge 43 on the anterior optic. In particular, the edge is located in the portion 43′ where the haptic couples to the anterior lens. As illustrated in FIG. 5B, the anterior-most portion 47 of haptic 46 is disposed approximately 0.5≦z≦0.8 mm more anteriorly located than an anterior side of edge 43 of the anterior optic (as measured when the AIOL is fully hydrated and in a saline bath with substantially no stress applied to the lens). Typically, the anterior-most portion of haptic 46 is approximately 0.75 mm more anteriorly located than the edge of the anterior optic. In some embodiments, the anterior-most portion of haptic 46 is approximately 0.6 mm more anteriorly located than the edge of the anterior optic.

As indicated above, anterior lens 42 and posterior lens 44 are coupled together by a plurality longitudinal haptics 46. The term “longitudinal” as used herein means extending arcuately along a surface of a substantially spherical or spheroidal object. In particular, where the object is a multi-element AIOL “longitudinal” means in a direction extending from the center of the anterior element to the center of the posterior element.

The haptics may be coupled to the peripheral edges of the anterior and posterior lenses by staking, integral formation, gluing, or other known techniques and the haptics are typically positioned around the peripheral edges of the lenses in equidistant peripheral locations. In a preferred embodiment, the haptics subtend an angle of thirty to forty degrees as viewed in a direction of line of sight (see FIG. 4), and extend outwardly approximately 4.65 mm from the optical axis (i.e., the overall diameter is approximately 9.3 mm in diameter), to approximate the normal internal diameter of the capsular bag of the human eye.

Anterior optic 42 is preferably bi-convex as depicted in FIG. 5A and is sized and shaped for positioning within a capsular bag 34. The power distribution of the anterior and posterior lenses may be selected to suit the needs of the particular patient, however, in a preferred embodiment, the anterior lens is positive and the posterior lens is negative. The optical axis OA of posterior lens 44 is in axial alignment with optical axis OA′ of anterior lens 42 and cooperates with the anterior lens to align with the visual axis VA of a patient's eye and to correct a wearer's vision. In a preferred embodiment, the lens 44 is fashioned in a spherical concavo-convex shape as depicted in FIGS. 3 and 5A. Although in a preferred embodiment the anterior and posterior lens combinations are as stated above, other lens couples are contemplated by the subject invention including anterior lens fashioned with concavo-planar, concavo-convex, and convex-concavo configurations. In a similar manner, the posterior lens may also exhibit the range of physical lens formation possibilities of being concave or convex or planar in order to achieve the desired visual result for a particular patient. It is to be appreciated that, in some embodiments, the accommodative effects of an AIOL may be achieved with the anterior lens having optical power and the posterior element having no optical power.

The haptics 46 are preferably composed of silicone material which can be molded along with one of the anterior or posterior lenses. Further details regarding the manufacture of a lens according to aspects of the present invention are given in U.S. patent application Ser. No. 10/954,322 filed on Sep. 30, 2004, by Graney, et al. which is hereby incorporated by reference in its entirety.

Although three longitudinally-extending haptics covering arcs of 30-40 degrees of longitude (at the anterior lens) each, such as shown in FIGS. 3-5A, constitute a preferred embodiment of the invention, other haptic arrangements of from two to five or more in number are envisioned and can be selected to satisfy the requirement of sufficient flexibility to provide the accommodated focusing of the lens system and simultaneous stiffness to maintain the axial position and orientation of the lens optics. Examples of suitable spring forces for an AIOL are given in a patent application titled ACCOMMODATIVE INTRAOCULAR LENS HAVING DEFINED AXIAL COMPRESSION CHARACTERISTICS Attorney Docket No. P03834P2 filed on even date herewith. Said application is hereby incorporated by reference in its entirety.

FIG. 6 is a schematic illustration of a portion of AIOL 40 positioned within a capsular bag 16. Zonules 27 extend peripherally about and are connected to the capsular bag 16. In a condition when the ciliary muscle (not shown) is relaxed and retracted peripherally outwardly, the zonules 27 will be tensioned outwardly which will pull the equator of capsular bag 16 into a configuration shown in FIG. 6. This position of the capsular bag causes the anterior and posterior lens 44 to be positioned near to one another to achieve distance vision.

Although not shown, it will be appreciated that, when the peripheral ciliary muscles are contracted, tension on the zonules 27 is lessened and the capsular bag assumes a condition for near vision. In a preferred embodiment, the subject intraocular lens system cooperates with the ciliary muscle, and zonules and capsular bag to permit anterior axial motion of the anterior lens with respect to the posterior lens of approximately 1.9 millimeters and a power correction of approximately 4 diopters.

Having thus described the inventive concepts and a number of exemplary embodiments, it will be apparent to those skilled in the art that the invention may be implemented in various ways, and that modifications and improvements will readily occur to such persons. Thus, the embodiments are not intended to be limiting and presented by way of example only. The invention is limited only as required by the following claims and equivalents thereto.

What is claimed is:

1. A multi-component accommodating intraocular lens (AIOL), comprising:
an anterior optical element;
a posterior component; and
at least two longitudinal haptics each coupled to at least a portion of the anterior optic and at least a portion of the posterior component, each haptic having an anterior-most portion that is disposed more anteriorly than a corresponding edge of the anterior optical element.

2. The AIOL of claim 1, wherein AIOL is sized and shaped to conform to the interior surface of a capsular bag of a patient's eye.

3. The AIOL of claim 2, wherein AIOL has an overall diameter of approximately 9.3 mm.

4. The AIOL of claim 1, wherein the anterior most-portion of each haptic is disposed greater than 0.5 mm more anteriorly than a corresponding edge of the anterior optical element.

5. The AIOL of claim 1, wherein the anterior most-portion of each haptic is disposed approximately 0.75 mm more anteriorly than the corresponding edge of the anterior optical element.

6. The AIOL of claim 1, wherein each of the haptics is integrally formed with the anterior optic.

7. The AIOL of claim 1, wherein the posterior component comprises an optical element.

8. The AIOL of claim 7, wherein the anterior optical element has a positive optical power and the posterior optical element has a negative optical power.

9. The AIOL of claim 1, wherein each of the haptics covers 30-40 degrees of longitude at the anterior lens.
